(19) United States
(12) Patent Application Publication
(10) Pub. No.: US 2022/0102003 A1
(43) Pub. Date: Mar. 31, 2022

(54) CASE PRIORITIZATION FOR A MEDICAL SYSTEM

(71) Applicant: Siemens Healthcare GmbH, Erlangen (DE)

(72) Inventors: Sven KOHLE, Erlangen (DE); Svenja LIPPOK, Uttenreuth (DE); Juergen SIMON, Wiesenthau (DE)

(73) Assignee: Siemens Healthcare GmbH, Erlangen (DE)

(21) Appl. No.: 17/480,289

(22) Filed: Sep. 21, 2021

(30) Foreign Application Priority Data
Sep. 30, 2020 (DE) 10 2020 212 318.7

(51) Int. Cl. G16H 50/20; G16H 10/60; G16H 40/20; G16H 70/20; G16H 70/60; G16H 10/40; G06F 3/14

(52) U.S. Cl. CPC G16H 50/20; G16H 10/60; G16H 40/20; G06F 3/14; G16H 70/60; G16H 10/40; G16H 70/20

(57) ABSTRACT

A computer-implemented method and apparatus are for processing medical cases. In an embodiment, the data set that is assigned to a medical case is received. A priority for processing the medical case is then determined for the medical case by applying to the data set trained functions that have been trained using training data sets and relevant known training priorities.

### PRIORITY STATEMENT

The present application hereby claims priority under 35 U.S.C. § 119 to German patent application number DE102020212318.7 filed Sep. 30, 2020, the entire contents of which are hereby incorporated herein by reference.

### FIELD

Example embodiments of the invention generally relate to methods for processing medical cases, and in particular to methods for the prioritized processing of a medical case by a technical system. Furthermore, example embodiments of the invention generally relate to a relevant apparatus, a medical system, a computer program and an electronically readable data carrier.

### BACKGROUND

In specialist medical fields, cases have to be prioritized for an evaluation since in many cases the starting of therapy is time-critical and this depends on a timely diagnosis. For example, prior to a pending evaluation in digital pathology, further data may have already been generated relating to the cases to be processed, such as for example radiology results. If radiology results are available, they may contain information that may provide an indication of how time-critical the evaluation of a case is in pathology. Other information such as lab results, anamnesis, tumor board minutes etc. can be used for the selection of a case, it being possible for the case to be prioritized both for the macroscopic pathological findings and for the microscopic pathological findings. Macroscopy (gross imaging) is the photographic recording of the entirety of the tissue removed (that is, for example, the whole of the tumor that has been removed), whilst in microscopy, sub-regions are viewed in colored form and in high resolution.

Cases are conventionally prioritized manually, based on information that the referring physician from a different specialist medical field passes on to the pathologist, based on the image information from the histopathology, or they are worked through according to the time of receipt, yet in such scenarios, time- and resource-critical cases may not be detected in time, with the result that bottlenecks may occur due to limited resources of a medical system, leading to cases not being processed in time and findings not being available in time.

### SUMMARY

The inventors have discovered that a need therefore exists for improved techniques for processing medical cases, which overcome or alleviate at least some of the aforementioned limitations and disadvantages.

The claims describe advantageous example embodiments of the invention.

Example embodiments of the invention are described hereinafter with reference to the method for which protection is sought, and also with reference to apparatuses for which protection is sought. Features, advantages or alternative example embodiments can be assigned in each case to a different claim category and vice versa. In other words, the claims for the apparatus may be improved by features that are described or for which protection is sought in the context of the methods. For example, the functional features of the method can be implemented by a computation apparatus of a medical system.

A computer-implemented method of an embodiment for processing cases comprises:

receiving a data set that is assigned to a medical case;

determining a priority for the medical case using the data set, comprising application of trained functions to the data set, the trained functions having been trained using training data sets and appropriate training priorities; and

providing the priority for a processing of the medical case.

A computer-implemented method of an embodiment for providing trained functions for determining a priority of a medical case comprises

receiving a training data set relating to at least one medical training case, and a known training priority for the at least one medical training case;

applying trainable functions to the training data set, wherein a priority for the medical training case is determined by applying the trainable functions to the training data set;

comparing the priority with the known training priority; and

adjusting at least one parameter in the trained functions, based upon the comparison of the priority with the known training priority.

An apparatus of an embodiment, comprises a computation unit, a memory unit, and an interface unit, wherein the memory unit stores executable commands from the computation unit, and wherein the apparatus is embodied when the commands are carried out in the computation unit to carry out at least:

receiving a data set that is assigned to a medical case that is to be processed by a medical system;

determining a priority for the medical case using the data set, comprising an application of trained functions to the data set, wherein the trained functions have been trained using training data sets and appropriate known training priorities; and

providing the priority for a processing of the medical case.

A computer program comprises commands which, when the program is carried out by a computer, cause the computer to carry out the steps of any desired method according to an embodiment of the present disclosure.

An electronically readable data carrier comprises commands which, when carried out by a computer, cause the computer to carry out the steps of an embodiment of any desired method according to the present disclosure.

A distributed database, in particular a cloud or a cloud application, comprises data sets and commands, which when the program is carried out by a computer, cause the computer to carry out the steps of an embodiment of any desired method according to the present disclosure.

A computer-implemented method, comprising:

receiving a data set assigned to a medical case;

determining a priority for the medical case using the data set, the determining including applying trained functions to the data set, the trained functions having been trained using training data sets and appropriate training priorities; and

providing the priority for a processing of the medical case.

A computer-implemented method of an embodiment, for providing trained functions for determining a priority of a medical case, comprises:

receiving a training data set relating to at least one medical training case, and receiving a known training priority for the at least one medical training case;

applying trainable functions to the training data set, wherein a priority for the at least one medical training case is determined by applying the trainable functions to the training data set;

comparing the priority with the known training priority; and

adjusting at least one parameter in the trained functions, based upon the comparing of the priority with the known training priority.

An apparatus of an embodiment, comprises:

computation circuitry;

a memory to store executable commands from the computation circuitry,

wherein the computation circuitry is embodied, upon the commands being carried out in the computation circuitry, to carry out at least:
receiving a data set assigned to a medical case to be processed by a medical system, and
determining a priority for the medical case using the data set, the determining of the priority including applying trained functions to the data set, the trained functions having been trained using training data sets and appropriate known training priorities; and
an interface to provide the priority for a processing of the medical case.

A non-transitory electronically readable data carrier of an embodiment stores commands which, when carried out by a computer, cause the computer to carry out the computer-implemented method of an embodiment.

### BRIEF DESCRIPTION OF THE DRAWINGS

The invention is described in greater detail hereinafter with reference to embodiments and the attached drawings.

FIG. 1 shows a flow diagram with steps for processing medical cases using a medical system according to some example embodiments.

FIG. 2 shows a flow diagram with steps for providing trained functions for determining a priority for a medical case, according to some example embodiments.

FIG. 3 shows in schematic form an apparatus with which a method according to the invention can be carried out according to some example embodiments.

The elements, features, steps and concepts referred to in the aforementioned will be obvious from the detailed description that follows by way of example embodiments, which are explained with reference to the attached drawings.

The drawings are to be regarded as schematic representations and the elements represented in the drawings are not necessarily shown true to scale. The various elements are rather shown such that their function and their general purpose become obvious to a person skilled in the art. Each connection or linkage between functional blocks, apparatuses, components or other physical or functional units that are described in the drawings or in the present document can also be achieved by an indirect connection or linkage. A linkage between the components can also be created with a wireless connection. Functions can be implemented in hardware, firmware, software or a combination thereof.

### DETAILED DESCRIPTION OF THE EXAMPLE EMBODIMENTS

The drawings are to be regarded as being schematic representations and elements illustrated in the drawings are not necessarily shown to scale. Rather, the various elements are represented such that their function and general purpose become apparent to a person skilled in the art. Any connection or coupling between functional blocks, devices, components, or other physical or functional units shown in the drawings or described herein may also be implemented by an indirect connection or coupling. A coupling between components may also be established over a wireless connection. Functional blocks may be implemented in hardware, firmware, software, or a combination thereof.

Various example embodiments will now be described more fully with reference to the accompanying drawings in which only some example embodiments are shown. Specific structural and functional details disclosed herein are merely representative for purposes of describing example embodiments. Example embodiments, however, may be embodied in various different forms, and should not be construed as being limited to only the illustrated embodiments. Rather, the illustrated embodiments are provided as examples so that this disclosure will be thorough and complete, and will fully convey the concepts of this disclosure to those skilled in the art. Accordingly, known processes, elements, and techniques, may not be described with respect to some example embodiments. Unless otherwise noted, like reference characters denote like elements throughout the attached drawings and written description, and thus descriptions will not be repeated. At least one embodiment of the present invention, however, may be embodied in many alternate forms and should not be construed as limited to only the example embodiments set forth herein.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “connected,” “engaged,” “interfaced,” and “coupled.” Unless explicitly described as being “direct,” when a relationship between first and second elements is described in the above disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

When an element is referred to as being “on,” “connected to,” “coupled to,” or “adjacent to,” another element, the element may be directly on, connected to, coupled to, or adjacent to, the other element, or one or more other intervening elements may be present. In contrast, when an element is referred to as being “directly on,” “directly connected to,” “directly coupled to,” or “immediately adjacent to,” another element there are no intervening elements present.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

Before discussing example embodiments in more detail, it is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments of the present invention. This invention may, however, be embodied in many alternate forms and should not be construed as limited to only the embodiments set forth herein.

Units and/or devices according to one or more example embodiments may be implemented using hardware, software, and/or a combination thereof. For example, hardware devices may be implemented using processing circuitry such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one embodiment of the invention relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

A computer-implemented method of an embodiment for processing cases comprises:

receiving a data set that is assigned to a medical case;

determining a priority for the medical case using the data set, comprising application of trained functions to the data set, the trained functions having been trained using training data sets and appropriate training priorities; and

providing the priority for a processing of the medical case.

In one step of an embodiment, data relating to one or a plurality of medical cases is received; in particular a data set that is assigned to a medical case is received. In some examples, a plurality of data sets can be received, with each of the plurality of data sets being able to be assigned to a different medical case in a multiplicity of medical cases.

In general therefore, data can be received or stored, it being possible for storage of data in a data memory and readout of data from a data memory to be comprised, and for a data memory to be able to comprise any desired internal or external, permanent data memory or main memory in a computation apparatus. For example, data can be received by a distributed database or a communications network and/or exchanged therewith, it being possible for data to be assigned to different technical systems of different specialist medical fields, that is, being able to originate therefrom.

In a further step of an embodiment, a priority for the medical case is determined, using the data set, comprising application to the data set of trained functions, in other words using a trained model. The trained functions are trained using training data sets and appropriate known training priorities. Therefore, by applying trained functions to the data set as an input, a priority, or a priority value, can be determined as an output. In this context, the trained functions can comprise end-to-end-trained functions or an end-to-end-trained model that contains a multiplicity of functional or model parameters, which have been trained based upon the application of the model to a training data set for determining a priority and for comparing the priority that has been determined with a training priority.

The determination of a priority can therefore contain a determination of a previous processing, that is, of an order, compared with a different medical case, or can contain a determination of a priority value for a medical case. A priority value relating to a medical case can be compared with a priority value relating to a different medical case in order to establish an order for processing the medical cases, or in other words, to select one of the cases for processing.

In a further step of an embodiment, the priority is provided for the processing of the medical case. In some examples, a medical case is therefore chosen or selected from a multiplicity of medical cases, in order to process it on a medical technical system with limited resources preferentially or prioritized, for example. In some examples, the medical case can be processed based on the priority.

The medical case can be processed in a first specialist medical field, and the data set can comprise data which originates from a specialist medical field that differs from the first specialist medical field. For example, the data can originate from at least one further technical system relating to a different specialist medical field. In some examples, the data can originate from a plurality of specialist medical fields that differ from the first specialist medical field, in particular from a plurality of technical examination systems in different specialist medical fields.

For example, the data set can comprise one or a plurality of or all, in particular any desired specific subset or any desired specific combination, of the following data and/or parameters of the medical case, or consist thereof:
a date of a forthcoming case conference, or a period of time up to a forthcoming case conference;
a manually determined parameter or comment from a referring physician;
a parameter that defines whether the time of evaluation is relevant to a decision on a diagnosis or therapy, with the parameter being determined by an application of trained functions to the data set relating to the medical case, with a training data set further containing reference data indicating whether the time of evaluation of the training case was critical for a decision on a diagnosis or therapy, in particular a manual note, indicating whether the training case should have been prioritized;
a parameter that defines whether a forthcoming tumor board or case conference is critically relevant for the patient for the success of a therapy, the parameter being determined by applying trained functions to the data set relating to the medical case, wherein a training data set further contains a reference datum indicating whether a forthcoming tumor board or case conference was critical for the training case for the success of the therapy;
a parameter that defines whether the processing is in connection with at least one previously determined (suspected) diagnosis;
a value from a lab test;
a pre-existing condition;
a pathology image of a pre-existing condition;
general patient data (e.g. age, weight, ICD codes, status, e.g. mobile/bedridden, covered by private/statutory insurance).

In some examples, based upon the data set, the trained functions can determine one or a plurality of parameters that define whether for the patient a forthcoming tumor board or case conference is relevant for the success of a therapy, and/or whether the time of evaluation is relevant for a decision on a diagnosis or therapy and can use these parameters for determining the priority. Relevant can be understood to mean that the event plays a part for the success or the decision, for example, that it is a key decisive factor (causal) or is the only decisive factor. Here, the trainable functions are trained based upon a multiplicity of other known medical cases (training cases), wherein the respective information is known for the training cases and is used for the training. Accordingly, the method can be applied to provide trained functions for the parameters described, by comparing the parameters determined by the trained functions with the reference information. The parameters described can also be generated by specific further trained functions, however, and provided for a determination of a priority.

At least one of the parameters can be an output value from a further trainable model that has been applied to a further data set from a different technical system, in particular from a different specialist medical field:
an automated image evaluation of available image data relating to the medical case, in particular radiological images (CT, MRI, PET/SPECT images, in particular of the body region involved in the biopsy), based on a trained model;
an automated Natural Language Processing (NLP), based on a trained model, of written documents pertaining to the medical case and/or the patient and/or voice recordings;
an automated analysis, based on a trained model, of a probable need for a follow-up examination on a medical system, and/or a beginning/change/end of a treatment/therapy.

A prioritization can therefore be determined for a specific technical system in a specialist medical field.

For example, using a further trainable model for a processing procedure that is to be carried out by the technical system, a resource consumption, or an estimated resource consumption of resources in the technical system by the medical case to be processed, or a resource consumption over time, can be determined. An output of the further model can therefore comprise a resource consumption of a medical case, that is, when processing the potential case on the technical system.

In particular, an available capacity or an availability of resources of the technical system or over time can be taken into account.

Therefore, an input parameter of the trained functions for determining a priority of a medical case can comprise, for example, an output from a trained model for determining consumption of resources, that is, determining a resource parameter or a time series of resource parameters, by a forthcoming processing of the medical case, or by a medical system. The consumption of resources can be determined, for example, by applying the trainable model itself, or a further trainable model, to the data set.

It is to be understood that any desired technique corresponding to the present disclosure can be restricted to any desired specific data set comprising one or a plurality of any desired specific combinations or subsets of the aforementioned data and/or parameters.

The trained functions can be trained based upon a manually set priority, or of a manual change in a priority previously determined by computer implementation, in particular based upon a comparison of a previous manual change in a priority with a priority determined by computer implementation. Therefore, the trained functions can be trained on a chronologically continuous basis to determine a priority during a use in a specialist medical field of the techniques according to the invention.

In a further step of an embodiment, a list can be displayed to a user, comprising the prioritized medical case together with further medical cases. Furthermore, at least one parameter that led to the prioritization of the medical case, that is, which was decisive in the prioritization, or a probability parameter (confidence value), which predicts how probable it is for the case to be prioritized, can be displayed for the prioritized medical case. An order of cases displayed may correspond to an order relating to the prioritization of the cases.

The techniques relating to the present disclosure can preferably be used in (digital) pathology, with the data set preferably containing data from radiology.

A method for providing trained functions for determining a priority of a medical case is provided hereinafter as a separate method that can be carried out independently of the method for determining a priority of a medical case using the trained functions.

In some examples, the method is used to provide the trained functions that are required in the method for determining a priority for a medical case, as a result of which the two methods complement each another and interact with each other, that is, correlate with each other and are dependent on each other.

A computer-implemented method of an embodiment for providing trained functions for determining a priority of a medical case comprises

receiving a training data set relating to at least one medical training case, and a known training priority for the at least one medical training case;

applying trainable functions to the training data set, wherein a priority for the medical training case is determined by applying the trainable functions to the training data set;

comparing the priority with the known training priority; and

adjusting at least one parameter in the trained functions, based upon the comparison of the priority with the known training priority.

A computer-implemented method of an embodiment for providing trained functions for determining a priority of a medical case comprises:

In one step of an embodiment, at least one training data set that is assigned to a medical training case is received. Furthermore, a known training priority (reference priority) is received for the medical training case.

In a further step of an embodiment, trainable functions that generate a priority by applying a priority to the data set are provided. The trainable functions are applied to the training data set, as a result of which a priority for the training case is determined.

In a further step of an embodiment, the priority that has been determined is compared with the reference priority, with a comparison comprising in particular a determination of a difference between the priority that has been determined and the reference priority.

In a further step of an embodiment, the trainable functions are trained based upon the comparison, in particular of the difference, with values for the parameters being adjusted, as a result of which an output of the trained functions corresponds to the known training priority. Through an optimization method, the difference between the output priority and the reference priority can be minimized.

It is to be understood that training of the trainable functions can advantageously be carried out using a multiplicity of training cases, it being possible for corresponding steps to be carried out for each of a multiplicity of training cases.

In some examples, the method for providing trained functions can be carried out continuously, or at recurring time intervals, or based upon a change in a data set or a manual change in a priority. For example, after processing of the medical case, it can be established whether a manual change in the prioritization has been carried out, and training can be carried out based upon the changed manual prioritization as a new known reference priority. In this sense, a processed medical case and the corresponding automatically determined priority, which has been confirmed by processing the case according to the automatically determined priority or by a manual confirmation, or which has been changed manually, can be used as the training data set for continuous training of the model.

The techniques disclosed therefore allow efficient utilization and planning of resources in a technical medical system, as a result of which relevant findings that have been established by the medical system are available more quickly and in a more reliable manner. A complex multiplicity of medical cases with different demands on resources can be better scheduled chronologically and/or an order of cases can be determined more efficiently, as a result of which bottlenecks in the resources of a medical system can be avoided. In particular, technical parameters of at least one different technical medical system in a different medical discipline can be used to determine a case priority, which further increases the efficiency of a selection according to a probable diagnosis and therapy.

Therefore, a corresponding medical system can be designed using fewer resources, as a result of which costs and hours of work can be reduced. At the same time, the quality of a diagnosis and therapy, and therefore patient safety, can be improved.

An apparatus of an embodiment comprises a computation unit, a memory unit, and an interface unit. The memory unit stores commands that are executable by the computation unit, the apparatus being embodied to carry out the steps of one of the methods in the present disclosure when carrying out commands in the computation unit.

A computer is configured to carry out a prioritization of medical cases. A computer can comprise, for example, a processor, a memory for storing program commands, and an interface for transmitting/receiving data. Here the memory stores the commands that are executable by the processor, with the computer carrying out the steps of any desired method or of any desired combination of methods according to the present disclosure when carrying out commands in the processor.

A technical system, in particular a medical technical system, is embodied to carry out the steps of any desired method according to the present disclosure. To this end, the medical system can comprise at least one apparatus according to the present disclosure.

A computer program comprises commands which, when the program is carried out by a computer, cause the computer to carry out the steps of any desired method according to an embodiment of the present disclosure.

An electronically readable data carrier comprises commands which, when carried out by a computer, cause the computer to carry out the steps of an embodiment of any desired method according to the present disclosure.

A distributed database, in particular a cloud or a cloud application, comprises data sets and commands, which when the program is carried out by a computer, cause the computer to carry out the steps of an embodiment of any desired method according to the present disclosure.

For such an apparatus, medical system, computer program, distributed database, and electronically readable data carrier, technical effects that correspond to the technical effects for the methods according to the present disclosure can be achieved.

Although the specific features that are described in the above summary and in the detailed description that follows are described in connection with specific examples, it is to be understood that the features can be used not only in the respective combinations but also in isolation or in any desired combinations, and features from different examples of the methods, apparatuses, medical systems, computer programs, distributed databases and electronically readable data carriers can be combined with one another and correlate with one another, insofar as it is not expressly stated otherwise.

It is to be understood that the techniques disclosed here are described both in connection with methods for applying trained functions, in other words one or a plurality of trained models, and with methods for providing appropriately trained functions that correlate with one another. Features, advantages or alternative example embodiments can be assigned to the other claimed methods and vice versa. In other words, claims for methods and systems providing trained functions can be improved by features that are described in connection with the methods and systems for applying trained functions and vice versa.

The above summary can therefore only provide a short overview of some features of some embodiments and implementations and is not to be understood as a restriction. Other embodiments can comprise features other than those described above.

Example embodiments are described in detail hereinafter with reference to the attached drawings. It has to be taken into account that the description that follows of the example embodiments is not to be understood in a narrow sense. The scope of the invention is not restricted by the example embodiments described hereinafter nor by the drawings, which merely serve to provide clarity.

Examples in the present disclosure relate to techniques for processing medical cases, for example by a medical technical system. Some examples relate to techniques for determining a priority, or a priority value, of a medical case, for processing the medical case on a medical system for example, to a determination of an order of two or a plurality of medical cases, to a determination or selection out of a multiplicity of medical cases of the next case to be processed, or in general, to methods for processing a medical case in a specialist medical field.

Pathologists want to prioritize their cases for evaluation because there are often some cases among them where starting therapy is time-critical, and this depends on the pathology results. Based upon the pathology images, it is difficult to predict which case should be prioritized, however. Prior to the pathological evaluation, further data, such as radiology results, for example, will have already been generated for each case. If radiology results are available, they may contain information that can provide an indication as to how time-critical the evaluation of a case is. Other information, such as lab results, anamnesis, tumor board minutes etc. can also be used for the prioritization. Here, the case can be prioritized both for the macroscopic pathological findings and for the microscopic pathological findings. Here macroscopy (gross imaging) is the photographic recording of the entirety of the tissue removed (that is, for example, the whole of the tumor that has been removed), whilst in microscopy, sub-regions are viewed in colored form and in high resolution.

Cases are conventionally prioritized manually, for example by carrying out an initial diagnosis, based upon information that the referring physician passes on to the pathologist, based upon the image information from the histopathology or they are worked through according to the time of receipt. Yet, a multiplicity of technical parameters available for a medical case from other technical systems, from other medical disciplines for example, are not taken into account since connections between these parameters with a priority of the case due to a high number of and complexity of causalities and correlations may not be known and cannot be detected manually. Therefore, a medical system cannot be utilized and operated efficiently; for example, at any desired given time the resources of the medical system may not be adequate if, for example, a less urgent case has been processed first, and a plurality of cases with higher priority must be processed at any desired subsequent time.

Some of the examples described here relate to a medical system in digital pathology, advantageously with data and/or parameters inter alia from a technical system in radiology being used; it is to be understood, however, that the techniques in the present invention can be used for processing or prioritizing cases on any desired medical technical system, that is, on a technical system in any desired medical discipline, it being possible for data from at least one further technical system to be used for a respective medical case.

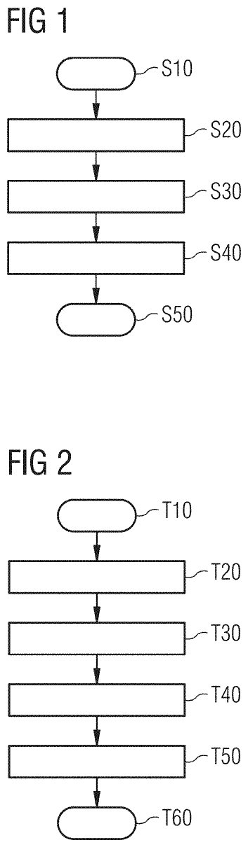

FIG. 1 shows a flow diagram with steps for processing medical cases using a medical system, according to some example embodiments.

The method begins in step S10. In step S20, a data set that is assigned to a medical case is received. In step S30, by applying trained functions to the data set, a priority for the medical case is determined, the trained functions having been trained using training data sets and appropriate known training priorities. In step S40, the priority for a processing of the medical case is provided. The method ends in step S50.

The techniques according to the disclosure can determine a prioritization in a machine-implemented manner, that is, automatically, based upon (that is, using) a set of rules/control parameters.

In such a scenario, there can be predetermined (combinations of) parameters that lead to a prioritization and are then clearly listed as the cause of the prioritization. Examples of such parameters are, for example, a date for a tumor board or for a different interdisciplinary case conference on the case that is taking place in less than two days, the age of a patient, the patient's insurance (private or statutory), flags/comments from referring physicians, for example the radiologist, seeking to prioritize this case.

In addition, there can be parameters that are learned from retrospective data as follows:

One parameter may be whether for this patient a timely tumor board or case conference was decisive for the success of a therapy. The simplest approach would be to obtain these notes from pathologists or oncologists (for example, by collecting cases where it was decisive or not decisive, based on a questionnaire or a software tool).

Alternatively, one could try to learn from the prioritization of case conferences. In each case, an input parameter for the algorithm is the note indicating whether such a case had been able to be prioritized: yes/no (I/O). The algorithm can receive standardized data sets for training in order to find patterns showing which parameters are relevant to the prioritization.

A further parameter can be whether the time of evaluation was critical: based on available training data, the algorithm can recognize features indicating that the time of evaluation in digital pathology was decisive for the subsequent therapy decision (for example, removal of the tumor only being successful up to a certain size of tumor, since due to rapid evaluation, the tumor had still been able to be removed in time before it spread) and/or was decisive for the success of the therapy (can be read off from the history/the patient management logfile, for example, and possibly the probability of survival can also be taken into account).

As an alternative to the change in therapy, a change of diagnosis could also be prepared for. A change in diagnosis can be determined by a comparison of documents in the patient file before and after the evaluation in digital pathology (NLP for free text, if necessary also ICD-10 codes if these are available).

A manual prioritization by the pathologist over time can be used to refine the algorithm, for example the order in which the pathologist processes the cases.

In principle, all the available data sets for a patient can be used to prioritize the individual cases.

Relevant input parameters, that is, input values for a prioritization, can be found by way of one or a combination of the following methods:

Basically, all the structured data relating to a patient (age, sex, weight, ICD codes, other case data) can be used without further pre-processing.

Machine-implemented Natural Language Processing (NLP) of (unstructured) written documents relating to the case and/or the patient, such as, for example, reports, letters from physicians and minutes of case conferences, such as, for example, tumor boards. By way of NLP, in particular (suspected) diagnoses can be determined and, advantageously, it can be established whether a current evaluation is associated with at least one of these diagnoses. In particular, a conclusion can then be drawn from the time-critical nature of the diagnosis regarding the priority of the evaluation.

Available radiological images (CT, MRI, PET/SPECT images, in particular of the body region involved in the biopsy) can be pre-processed using algorithms for image evaluation of the radiological images, in particular using algorithms based on machine learning. In particular, the properties of tissue or structures can be analyzed and classified using these algorithms.

Advantageously, an additionally issued confidence value can also be used for such an algorithm. This confidence value corresponds to the certainty with which an output value from the algorithm actually corresponds to reality. In particular, higher priority can be given to evaluations where there is low confidence regarding the radiological images (that is, a high degree of uncertainty, which can be reduced via the pathology).

Automated analysis of the probable need for further coloring procedures and/or molecular pathology and/or of the need for further examinations to confirm the final diagnosis. The final decision on further coloring procedures is taken by the pathologist, but the parameter may be relevant for the prioritization.

The pathologist automatically receives a prioritized list of cases, which they can access manually as required. Where possible, the reason why a case has been prioritized can be visually displayed. This is possible if the parameters that led to prioritization are known. For the other cases, a probability parameter that predicts the probability of a case needing to be prioritized can be displayed.

The cases can be displayed in a list of high to low priority. Particularly critical cases can be emphasized by a symbol/color etc. In particular, information indicating when a tumor board for the respective case is taking place can be displayed to the pathologist if this has been fixed.

It is not only information from pathology that is used, but all or a selection of the available data relating to the case, which makes it more probable that the correct cases are prioritized. Urgent diagnoses can therefore be prioritized and on average reach the referring physician more quickly. Since the pathology results often constitute the bottleneck at the end of the chain of evaluations, patients can therefore receive more quickly the therapy that is time-critical for them, as a result of which the prognoses for patients can improve. Case conferences can more often make their decisions based upon the necessary results data. The use of image data can ensure that even such data that the radiologist classifies as supposedly not relevant to prioritization is taken into account in the prioritization of cases in pathology.

FIG. 2 shows a flow diagram with steps for providing trained functions for determining a priority for a medical case, according to some example embodiments.

The method begins in step T10. In step T20, a training data set from at least one medical training case is received, and a known priority for the at least one medical training case, the processing of the medical training case for example, is further received. In step T30, trainable functions are applied to the training data set, a priority being determined for the medical training case by applying the trainable functions to the training data set. In step T40, the priority determined for the medical training case is compared with the known training priority. Based upon the comparison, in step T50, at least one parameter that is contained in the trainable functions is adjusted; in other words, the trainable functions are trained based upon the training data set and the training priority. The method ends in step T60.

The techniques described therefore have the effect that resources of a technical system can be utilized more efficiently, that is, resources of a technical system can be more precisely distributed timewise by a (continuing) determination/selection of cases for processing being facilitated through the use of technical parameters and/or output values and/or signal values and/or measured values from at least one further technical system of a different medical discipline, that is, from a different specialist medical field, for example in a way that a bottleneck in a resource of a medical system that is to be used does not occur at any later time. Here, the scheduling of cases according to their urgency is guaranteed, such that the relevant technical system with fewer resources can complete the upcoming processing tasks on time and economically.

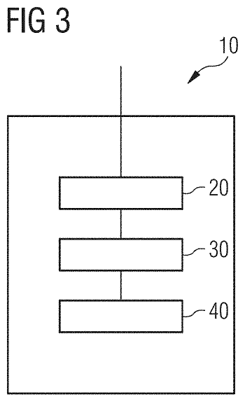

FIG. 3 shows in schematic form an apparatus 10, with which a method according to the invention can be carried out according to some example embodiments.

The apparatus 10 comprises a computation unit 30, a memory unit 40, an interface unit 20, wherein the memory unit 40 stores commands that can be executed by the computation unit 30, and wherein the apparatus 10 is embodied to carry out the following steps of a method according to the present disclosure when the commands are carried out in the computation unit 30.

A computation unit, or processor, can be understood in connection with embodiments of the invention to mean, for example, a machine or an electronic circuit. A processor can be in particular a central processing unit (a CPU), a microprocessor, or a microcontroller, for example an application-specific integrated circuit or a digital signal processor, possibly in combination with a memory unit for storing program commands etc. A processor can also be, for example, an IC (integrated circuit), in particular an FPGA (field programmable gate array) or an ASIC (application-specific integrated circuit), or a DSP (digital signal processor) or a GPU (graphic processing unit). A processor can also be understood to mean a virtual processor, a virtual machine or a soft CPU. It can also be, for example, a programmable processor that is equipped with configuration steps for carrying out the aforementioned method according to embodiments of the invention or that is configured with configuration steps such that the programmable processor implements the features according to embodiments of the invention of the method, the component, the modules, or other aspects and/or sub-aspects of embodiments of the invention.

A memory, a memory unit or memory module and suchlike can be understood in the context of embodiments of the invention to be, for example, a volatile memory in the form of a random-access memory (RAM) or a permanent memory such as a hard drive or a data carrier.

In general, examples of the present disclosure provide a multiplicity of circuits, data memories, interfaces or electrical processing apparatuses such as processors. All references to these units and other electrical devices together with the functions provided thereby are not restricted to what is illustrated and described. While certain terms can be assigned to the various circuits or other electrical devices disclosed, these terms are not intended to restrict the functional scope of the circuits and of the other electrical devices. These circuits and other electrical devices can be combined with one another and/or separated from one another according to the respective desired type of electrical design. It is to be understood that each disclosed circuit or other electrical apparatus can comprise any desired number of microcontrollers, graphic processor units (CPUs), integrated circuits, memory apparatuses such as flash disks, main memory (RAM), read-only memory (ROM), electrically programmable read-only memory (EPROM), electrically erasable programmable read-only memory (EEPROM), or any desired other embodiments of the same, such as software, which work together to carry out the process steps disclosed herein. In addition, each electrical apparatus can be configured to carry out a program code which is contained in an electronically readable data carrier, and which is configured to carry out any desired number of steps according to the method in the present disclosure.

Some general conclusions can be drawn from what was the in the aforementioned:

The methods can preferably be achieved in a computer-assisted manner, that is computer-implemented and/or automated.

The application of trained functions can be carried out by a neural network, which can comprise a multiplicity of classification functions. In various examples, the trained functions can comprise one or a plurality of known classifiers for machine learning. Without restrictions, the trained functions can be based, for example, on one or a plurality of a support-vector-machine, a decision tree and/or a Bayesian network, k-means clustering, Q-learning, genetic algorithms and/or association rules. A neural network can be for example a deep neural network, a convolutional neural network or a convolutional deep neural network, an adversarial network, a deep adversarial network and/or a generative adversarial network or a model-based machine-learning network architecture.

An AI engine or a computation module in a medical system can be configured to carry out one of the methods described and can use at least one machine learning function or classifier known in the technology, such as, for example, SVM and/or a neural network. In various examples, the AI engine uses a multiplicity of machine learning functions, for example seven or more machine learning functions in a stratified network architecture.

For processing the parameters, neural networks and support vector machines can be used, for example. To analyze the sensor data, sequential qualification algorithms such as for example an LSTM can be used. With regard to the determination of the SOH, the application of trained functions to parameters and/or raw sensor data, that is, measured data from the loading process, can comprise the application of at least one classification algorithm to the input data, which algorithm can involve a classifier for machine learning. The trained functions can comprise, for example, machine-trained classifiers, which are applied to a data set in a plurality of strata in a neural network.

Trained functions can include a trainable algorithm or a trainable model that can be applied to the parameters and/or to the measured data. For example, trained functions can comprise a multiplicity of model parameters that define how the trained functions are applied to the parameters and measured data, and how an age can be determined from the parameters and/or measured data. Here the model parameters can be adjusted, corrected, or changed based upon the application of the trained model to the parameters and/or measured data, such that the reparametrized model can be used to determine a precise prioritization.

A priority value of the medical case can therefore be used for the prioritization or selection of the medical case. For example, the medical cases can be written into an ordered data set, based on the priority values.

The data set can include data, measured data, and parameters that have been determined from the measured data, in particular the data can be live data, such as live measured data measured continuously or continually or in real time, for example, at regular intervals and/or during and/or after the method for determining a priority. In general, parameters can comprise discrete values of two states 0 or 1, that is, a flag, or a plurality of discrete values, or one or a plurality of discrete values from a continuous range of values.

In some examples, the data set can comprise data from 2, or 3, or 4, or 5 different specialist medical fields. Such specialist fields can include specialist fields of human medicine, and/or dentistry, or veterinary medicine.

A determination of a priority can in general comprise a determination of a comparative parameter (priority value) for comparison with a different medical case based on the comparative parameter, and accordingly a case can be chosen or selected or prioritized out of a multiplicity of cases, based upon the comparative parameter. A case can therefore be prioritized with respect to a second case; in other words an order or a time for processing can be determined in comparison with a second case, such that a case can be processed using the comparative value.

Provision of the prioritization of a medical case can comprise listing of cases in an order that corresponds to the prioritization.

Techniques according to the present disclosure can be carried out based upon a trigger, which can be set manually or automatically. For example, the methods can be triggered to prioritize cases to be processed by a predetermined event in a technical system of a different medical discipline, for example, a time for the measurement of further data in the data set, or can be carried out continually, in each case after a predetermined time interval. It is also conceivable for a corresponding time interval or an implementation time to be determined continually based upon the data set and dynamically using a trained model.

Providing a prioritization can further comprise providing a confidence value for a prioritization.

Parameters can include time series of parameters, that is, at least one parameter in the data set can comprise a plurality of measured values with the appropriate times.

A data set for a medical case can be stored in a distributed database, which is implemented in a communications network of the technical systems for different specialist medical fields.

Prioritization of one or of a plurality of cases can ensue regularly after a predetermined time interval or can then be carried out for a case if a relevant data set has been updated. Therefore, an updated order of the cases to be processed, which can represent a current data situation, such as an emergency case, for example, can be determined continually.

By prioritizing one or a plurality of medical cases against one another, an order of medical cases for processing can be established.

In some examples, the method can be applied to all the medical cases to be prioritized, it being possible, for example, for a prioritization to be established in pairs of two cases, comparing one with the other. In other examples, the method can be applied to one case only, to a new case or to a case with a changed data set, wherein a changed priority value can be determined. Based upon the priority value, a prioritization or order of medical cases for processing can be determined, for example a medical case can be classified in an existing order of cases. Therefore, resources of the technical system, the availability of which may vary at times, can be used more efficiently.

The application of trained functions to a data set can comprise the output of a priority value and of a relevant confidence value, both values being generated by the application of trainable functions to the data set, or can comprise the determination of a priority of a case out of a multiplicity of cases.

As further data, the data set can comprise parameters and/or examination results from a different medical discipline. Any desired or all the parameters/data can preferably be provided with a time value, for example a time stamp that indicates a time or a timescale at which the data/parameters have been determined or acquired.

Processing of the medical case can also comprise a case conference for the medical case, for example.

A technical system can be a medical system that is required for examining a specimen from a patient, for example, or for establishing a diagnosis.

The data set can include patient data that characterizes the progression of the medical case, for example earlier examinations, progression data, that is data on the progression over time of a parameter that changes over time; other conceivable data might be data relating to a stay in hospital, for example ward/intensive care ward and similar data relating to treatment units.

A method can comprise a determination of a priority value for each of a multiplicity of medical cases, wherein furthermore one of the cases is selected and/or processed by the technical system.

The method can further comprise display on a display unit of at least the medical case to be prioritized, wherein the prioritized cases are displayed to the user according to the prioritized order thereof, and wherein in each case at least one of the parameters that led to the prioritization of the case and/or a confidence value for the prioritization is additionally displayed.

In summary, techniques for the processing of medical cases by a technical system are provided, wherein the medical case is processed in various specialist medical fields, for example using various technical systems. A trained model that has been trained using the data from known medical cases is applied to a data set that is assigned to a medical case. The data set comprises the data currently available for the medical case, in particular data from different specialist medical fields. By applying the model, a priority for the processing of the medical case can be determined automatically, which allows in a computer-assisted method the resources of the technical system to be distributed selectively and at optimized times to cases according to their priority. As a result thereof, bottlenecks in resources can be avoided and the technical system can be designed with fewer resources. Accordingly, the quality and temporal availability of the evaluation using a medical system and hence patient safety can be improved.

Although the invention has been demonstrated and described with reference to certain preferred embodiments, equivalents and changes are made by persons skilled in the art after reading and understanding the description. The present invention comprises all such equivalents and changes and is restricted only by the scope of the attached claims.

Although the invention has been illustrated and described in detail by the preferred embodiments, the invention is not limited by the disclosed examples and other variations can be derived herefrom by the person skilled in the art without departing from the scope of protection of the invention.

Even if not explicitly stated, individual example embodiments, or individual sub-aspects or features of these example embodiments, can be combined with, or substituted for, one other, if this is practical and within the meaning of the invention, without departing from the present invention. Without being stated explicitly, advantages of the invention that are described with reference to one example embodiment also apply to other example embodiments, where transferable.

Of course, the embodiments of the method according to the invention and the imaging apparatus according to the invention described here should be understood as being example. Therefore, individual embodiments may be expanded by features of other embodiments. In particular, the sequence of the method steps of the method according to the invention should be understood as being example. The individual steps can also be performed in a different order or overlap partially or completely in terms of time.

The patent claims of the application are formulation proposals without prejudice for obtaining more extensive patent protection. The applicant reserves the right to claim even further combinations of features previously disclosed only in the description and/or drawings.

References back that are used in dependent claims indicate the further embodiment of the subject matter of the main claim by way of the features of the respective dependent claim; they should not be understood as dispensing with obtaining independent protection of the subject matter for the combinations of features in the referred-back dependent claims. Furthermore, with regard to interpreting the claims, where a feature is concretized in more specific detail in a subordinate claim, it should be assumed that such a restriction is not present in the respective preceding claims.

Since the subject matter of the dependent claims in relation to the prior art on the priority date may form separate and independent inventions, the applicant reserves the right to make them the subject matter of independent claims or divisional declarations. They may furthermore also contain independent inventions which have a configuration that is independent of the subject matters of the preceding dependent claims.

None of the elements recited in the claims are intended to be a means-plus-function element within the meaning of 35 U.S.C. § 112(f) unless an element is expressly recited using the phrase “means for” or, in the case of a method claim, using the phrases “operation for” or “step for.”

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A computer-implemented method, comprising:
receiving a data set assigned to a medical case;
determining a priority for the medical case using the data set, the determining including applying trained functions to the data set, the trained functions having been trained using training data sets and appropriate training priorities; and
providing the priority for a processing of the medical case.

2. The computer-implemented method of claim 1, wherein the data set includes data for at least one further technical system in a different specialist medical field, the data being data assigned to the medical case.

3. The computer-implemented method of claim 1, wherein the data set comprises at least one of:
a date of a forthcoming case conference, or a period of time leading up to a forthcoming case conference;
a manually determined parameter or comment from a referring physician;
a parameter defining whether a time of evaluation is relevant to a decision for a diagnosis or therapy, wherein the parameter is determined by applying trained functions to the data set for the medical case, wherein a training data set further contains reference information as to whether the time of evaluation of the training case was critical for a decision on a diagnosis or therapy;
a parameter defining whether, for the patient, a timely tumor board or case conference is critically relevant to the success of a therapy, wherein the parameter is determined by applying trained functions to the data set of the medical case, wherein a training data set further contains reference information indicating whether, for the training case, a timely tumor board or case conference was critical for the success of the therapy;
a parameter defining whether the processing is in connection with at least one previously determined diagnosis;
a value from a lab test;
a pre-existing condition;
a pathology image of a pre-existing condition; and
general patient data.

4. The computer-implemented method of claim 1, wherein the trained functions have been trained based upon a comparison of a previous manual change in a priority with a priority determined by computer implementation.

5. The computer-implemented method of claim 1, wherein at least one of the parameters is an output value from a further trainable model that has been applied to a data set from a further technical system, comprising at least one of:
an automated image evaluation, based on a trainable model, of available image data relating to the medical case;
machine-implemented Natural Language Processing (NLP), based upon a trainable model, of written documents or voice recordings pertaining to at least one of the medical case and the patient; and
a machine-implemented determination, based upon a trainable model, of at least one of a probable need for a follow-up examination on a medical system, and a beginning of or of a change in a treatment or a therapy.

6. The computer-implemented method of claim 1, further comprising:
displaying an ordered list comprising the medical case once prioritized, and further medical cases, in an order corresponding to prioritization.

7. The computer-implemented method of claim 1, wherein processing of the medical case has been carried out in pathology, and wherein the data set contains data from radiology.

8. A computer-implemented method for providing trained functions for determining a priority of a medical case, comprising:
receiving a training data set relating to at least one medical training case, and receiving a known training priority for the at least one medical training case;
applying trainable functions to the training data set, wherein a priority for the at least one medical training case is determined by applying the trainable functions to the training data set;
comparing the priority with the known training priority; and
adjusting at least one parameter in the trained functions, based upon the comparing of the priority with the known training priority.

9. An apparatus, comprising:
computation circuitry;
a memory to store executable commands from the computation circuitry,
wherein the computation circuitry is embodied, upon the commands being carried out in the computation circuitry, to carry out at least:
receiving a data set assigned to a medical case to be processed by a medical system, and
determining a priority for the medical case using the data set, the determining of the priority including applying trained functions to the data set, the trained functions having been trained using training data sets and appropriate known training priorities; and
an interface to provide the priority for a processing of the medical case.

10. A medical system, comprising at least one apparatus of claim 9.

11. The computer-implemented method of claim 3, wherein the training data set further contains reference information as to whether the time of evaluation of the training case was critical for a decision on the diagnosis or therapy based upon a manual note on whether the training case should have been prioritized.

12. The computer-implemented method of claim 2, wherein the trained functions have been trained based upon a comparison of a previous manual change in a priority with a priority determined by computer implementation.

13. The computer-implemented method of claim 2, wherein at least one of the parameters is an output value from a further trainable model that has been applied to a data set from a further technical system, comprising at least one of:
an automated image evaluation, based on a trainable model, of available image data relating to the medical case;
machine-implemented Natural Language Processing (NLP), based upon a trainable model, of written documents or voice recordings pertaining to at least one of the medical case and the patient; and
a machine-implemented determination, based upon a trainable model, of at least one of a probable need for a follow-up examination on a medical system, and a beginning of or of a change in a treatment or a therapy.

14. The computer-implemented method of claim 2, further comprising:
displaying an ordered list comprising the medical case once prioritized, and further medical cases, in an order corresponding to prioritization.

15. The computer-implemented method of claim 6, further comprising:
displaying at least one of
at least parameter relating to the medical case once prioritized, which led to the prioritization, and
a probability parameter predicting how probable for the medical case to have to be prioritized.

16. The apparatus of claim 9, wherein the computation circuitry includes at least one processor.

17. The apparatus of claim 9, wherein the computation circuitry includes at least one integrated circuit.

18. A non-transitory electronically readable data carrier storing commands which, when carried out by a computer, cause the computer to carry out the computer-implemented method of claim 1.

19. A non-transitory electronically readable data carrier storing commands which, when carried out by a computer, cause the computer to carry out the computer-implemented method of claim 8.
